(19) United States
(12) Patent Application Publication
(10) Pub. No.: US 2019/0183752 A1
(43) Pub. Date: Jun. 20, 2019

(54) AGENT FOR CHANGING THE COLOR OF KERATIN-CONTAINING MATERIALS HAVING AT LEAST ONE ALCOHOL, A MODIFIED CLAY MINERAL AND A COLOR PIGMENT

(71) Applicant: Henkel AG & Co. KGaA, Duesseldorf (DE)

(72) Inventors: Konstantin Goutsis, Juechen (DE); Daniela Kessler-Becker, Leverkusen (DE)

(73) Assignee: Henkel AG & Co. KGaA, Duesseldorf (DE)

(21) Appl. No.: 16/216,219

(22) Filed: Dec. 11, 2018

(30) Foreign Application Priority Data
Dec. 14, 2017 (DE) 10 2017 222 725.7

(51) Int. Cl. A61K 8/26; A61K 8/34; A61K 8/37; A61K 8/81; A61Q 5/06

(52) U.S. Cl. CPC A61K 8/26; A61K 8/345; A61K 8/37; A61K 8/8158; A61Q 5/065; A61K 2800/87; A61K 2800/651; A61K 2800/621; A61K 2800/612; A61K 2800/5922; A61K 2800/436

(57) ABSTRACT

The present disclosure relates to cosmetic agents for changing the color of keratinic materials, in particular human hair, containing
(a) at least one aliphatic and/or aromatic alcohol having from about 2 to about 8 carbon atoms,
(b) at least one clay mineral modified with quaternary ammonium compounds and
(c) at least one color pigment.

### CROSS-REFERENCE TO RELATED APPLICATION

This application claims priority to German Patent Application No. 10 2017 222 725.7, filed Dec. 14, 2017, which is incorporated herein by reference in its entirety.

### TECHNICAL FIELD

The present application is in the field of cosmetics and relates to the temporary color change of keratinic materials, in particular human skin and/or human hair. The subject of the application is cosmetic agents containing at least one aliphatic and/or aromatic alcohol, at least one clay mineral modified with quaternary ammonium compounds and at least one color pigment.

A further subject of the present application is also a method for the temporary dyeing of keratinic materials, in particular human skin and/or human hair, in which the aforementioned agent is used by employing a spray application.

### BACKGROUND

Changing the color of keratinic surfaces or materials, in particular human skin and/or hair, is an important area of modern cosmetics. The person skilled in the art knows various dyeing systems to change hair color, depending on the requirements of the dyeing. Oxidation colorants are used for permanent, intense dyeings having good fastness properties and good gray coverage. Such colorants usually contain oxidation dye precursors, so-called developer components and coupler components which, under the influence of oxidizing agents such as hydrogen peroxide, form the actual dyes with each other. Oxidation colorants are exemplified by very long lasting coloring results.

Ready-formed dyes diffuse from the colorant into the hair fiber when direct dyes are used. The dyeings obtained with direct dyes have a lower durability and faster leachability in comparison with the oxidative hair dyeing. Dyeings with direct dyes usually remain on the hair for a period of between from about 5 and about 20 washes.

Various methods are known in the prior art for changing skin color, in particular for the tanning of the skin or for the reduction of unwanted age spots. For example, the tanning of the skin may be performed using dyes which penetrate the skin layers and cause sustained browning. Active ingredients which penetrate the skin and decompose the age-related melanin accumulations are used to reduce age spots. Here, too, a long-lasting change in the skin color, which cannot or only with difficulty be removed by cleaning the skin, can be achieved.

In the context of modern fashion trends, however, there is also the need for color effects, which remain on the hair and/or skin only for a short period of time and then can be completely removed by one or more washes with surfactant-containing cleaning agents without leaving any residue from the hair and/or the skin. However, the color effect should be retained on contact with water or perspiration in order to avoid the undesirable running of the color due to rain or perspiration. Direct dyes diffuse more or less strongly into the hair fiber or the skin surface and remain there with several washes of surfactant-containing cleaning agents. This class of dyes is therefore not well suited for residue-free removal of the color effect after a single wash with surfactant-containing cleaning agents.

The use of color pigments is known for short-term color changes on the hair and/or skin. Color pigments are generally understood to be insoluble coloring substances. These are undissolved in the form of small particles in the coloring formulation and are deposited only on the outside on the hair fibers and/or the skin surface. Therefore, they can be removed without residue by a few washes with surfactant-containing cleaning agents. Various products of this type are available on the market under the name of hair mascara.

Depending on the desired dyeing effect, the content of color pigments in the agent is chosen very differently. Especially for the achievement of intense, fashionable and strong color effects, it may be necessary to use color pigments in amounts of over about 10% by weight, in some cases even up to about 30% by weight, in the agents. At the same time, the hair mascaras, in particular when they are provided in the form of a spray application, are regularly formulated as suspensions with relatively low viscosity. Products of this type can have a great disadvantage in that the color pigments contained in them sink during storage, so that the color pigments assemble into a compact layer at the bottom of the application container. Depending on the storage conditions and storage periods, this sediment of color pigments that has formed on the bottom can become so solid that it can no longer be loosened by the consumer through mere shaking. Products in this state can no longer be used by the user and are therefore highly undesirable.

### BRIEF SUMMARY

The object of the present disclosure is therefore to provide novel agents for changing the color of keratinic materials, in particular of hair and the skin, which do not have the aforementioned disadvantages. The agents should enable the creation of strong, fashionable and eye-catching color effects by employing the use of color pigments. The application of the agent should be possible by employing spray application. In addition, the agents should be stable in storage even with a high color pigment content, that is, the sedimentation of the color pigments should be prevented to the extent that the agents remain ready for use even after long storage periods through a brief shaking. At the same time, the agents should have good water resistance, perspiration resistance and rain resistance. This should prevent unplanned washing by rain or perspiration, planned removal of the agent through washing with a surfactant-containing cleaning agent should be possible.

Surprisingly, it has been found that these objects can be achieved by the use of color pigments and certain clay minerals modified with quaternary ammonium when used in a specific alcoholic carrier. The use of the modified clay minerals led to a considerable improvement in the storage stability of the agents, without, however, negatively influencing the coloring properties of these agents. In addition, the dyeing was removed without residue by employing some intensive washes with surfactant-containing cleaning agents and therefore leads, in contrast to a dyeing with oxidation dyes and direct dyes or bleaching with oxidizing agents, only to a temporary dyeing of the keratinic surfaces, in particular the skin and/or hair.

A first subject of the present disclosure is an agent for changing the color of keratinic materials, in particular human hair and/or skin, containing
(a) at least one aliphatic and/or aromatic alcohol having from about 2 to about 8 carbon atoms,
(b) at least one clay mineral modified with quaternary ammonium compounds, and
(c) at least one color pigment.

In other words, a first subject of the present disclosure is an agent for the temporary changing of color of keratinic materials, in particular human hair and/or skin, containing
(a) at least one aliphatic and/or aromatic alcohol having from about 2 to about 8 carbon atoms,
(b) at least one clay mineral modified with quaternary ammonium compounds, and
(c) at least one color pigment.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

### Agent for Changing the Color of Keratinic Material

Keratinic material is understood to mean keratinic fibers and the skin. Keratinic fibers, keratin-containing fibers or keratin fibers are understood to mean furs, wool, feathers and, in particular, human hair.

When skin is dyed, in particular human skin, in principle, all locations of the skin can be dyed, such as the skin on the décolleté, on the arms and legs, the scalp or the skin on the face.

The term “agent for changing the color” is used in the context of this present disclosure for a coloring of the keratin materials caused by use of color pigments, in particular the hair and/or the skin, wherein the color pigments, together with the other constituents essential to the present disclosure, are deposited as a film on the surface of the keratin material. Due to the hydrophobic properties of the formed film, the dyeings produced have a good water resistance and rain resistance, so that the dyeings survive the time until the next hair wash without loss of color. The user, however, can completely remove the dyeings from the hair again by intensive shampooing with a surfactant-containing cleaning agent. The term “agent for the changing of the color” used as contemplated herein is therefore understood to mean in particular “agent for the temporary changing of the color”. In the context of the present disclosure, an oxidative dyeing carried out with oxidation dyes is not included under the term changing the color. Also not included under the term temporary changing of the color is a brightening, bleaching or blonding of the keratin fibers caused by the use of an oxidizing agent. Neither the effect caused by the oxidative color change nor the bleaching effect can be reversed by shampooing, so both color changes are not temporary.

Unless specified otherwise, all specifications in % by weight relate to the total weight of the cosmetic agents as contemplated herein, wherein the sum of all ingredients of the agents as contemplated herein is about 100% by weight. If the cosmetic agents as contemplated herein contain at least one propellant, the specification % by weight refers to the total weight of the cosmetic agent, including the propellant present.

(a) Aliphatic and/or Aromatic Alcohols having from about 2 to about 8 Carbon Atoms

The agents as contemplated herein include at least one aliphatic and/or aromatic alcohol having from about 2 to about 8 carbon atoms as the first ingredient (a) essential to the present disclosure.

Aliphatic and/or aromatic alcohols having from about 2 to about 8 carbon atoms are compounds which have from about 2 to about 8 C atoms, are aliphatic and/or aromatic in nature and carry one or more hydroxyl groups.

For the purposes of the present disclosure, the alcohols (a) do not carry any heteroatoms other than oxygen. They may contain, in addition to the hydroxy group or groups, an ether grouping, but in addition have no functional groups other than the hydroxy group (that is, monoethanolamine, alpha-hydroxycarboxylic acids, dihydroxyacetone, etc. are not alcohols for the purposes of the present disclosure).

Suitable aliphatic alcohols are, for example, ethanol, isopropanol, n-propanol, butanol, n-pentanol, 1,2-propanediol, 1,3-propanediol, 1,3-butanediol, 1,4-butanediol, 1,2-hexanediol, 1,6-hexanediol and glycerol. Suitable aromatic alcohols are, for example, benzyl alcohol, phenoxyethanol and phenylethyl alcohol.

In an exemplary embodiment, an agent as contemplated herein for changing the color of keratinic materials contains one or more alcohols (a) from the group including of ethanol, isopropanol, n-propanol, butanol, n-pentanol, 1,2-propanediol, 1,3-propanediol, 1,3-butanediol, 1,4-butanediol, 1,2-hexanediol, 1,6-hexanediol, glycerol, benzyl alcohol, phenoxyethanol and phenylethyl alcohol.

The alcohols (a) as contemplated herein are organic solvents which suspend or disperse the other constituents (b) and (c) essential to the present disclosure and volatilize after application to the keratinic materials, in particular the hair and/or the skin. Due to the evaporation of the solvent, a film forms on the keratin fibers, which film is exemplified by outstanding water resistance and rain resistance.

It has been found that this film formation goes on well and uniform when the alcohol or alcohols are present in the agent in a minimum amount of at least about 25.0% by weight, for example of at least about 30.0% by weight, for example of at least about 35.0% by weight, for example of at least about 40.0% by weight and for example of at least about 50.0% by weight. The best results were observed with an alcohol amount of at least about 50% by weight. All amounts specified in % by weight here are based on the total amount of all alcohols (a) as contemplated herein, which is related to the total weight of the agent.

In an exemplary embodiment, an agent as contemplated herein contains, based on its total weight, one or more alcohols (a) in a total amount of at least about 25.0% by weight, for example of at least about 30.0% by weight, for example of at least about 35.0% by weight, for example of at least about 40.0% by weight and for example of at least about 50.0% by weight.

In an exemplary embodiment, an agent as contemplated herein contains, based on its total weight, one or more alcohols (a) in a total amount of about 25.0% by weight to about 90% by weight, for example of at least about 30% by weight to about 90% by weight, for example from at least about 35.0% by weight to about about 90% by weight, for example from at least about 40.0% by weight to about 90% by weight, and for example from at least about 50.0% by weight to about 85% by weight.

The alcohols from group (a) are compounds which have different boiling points and are of greatly different degrees of volatility. It has been found that ethanol has the best suitability within this group. When the agents contain significant amounts of ethanol, the color pigments (c) deposited on the keratin material form a film in which the color pigments (c) are distributed uniformly on the keratinic materials. The drying of the applied agent was also very fast, so that the abrasion of the not yet dried agent by friction on textiles could be minimized.

In an exemplary embodiment, an agent as contemplated herein contains, based on its total weight, (a) at least about 30.0% by weight, for example at least about 40.0% by weight, for example at least about 50.0% by weight, and for example at least about 60.0% by weight of ethanol.

In a further exemplary embodiment, an agent as contemplated herein contains, based on its total weight, (a) from about 30.0 to about 90.0% by weight, for example from about 40.0 to about 90.0% by weight, for example from about 50.0 to about 85.0% by weight and for example from about 60.0 to about 85.0% by weight of ethanol.

The properties described above can be still further improved when a smaller amount of polyhydric alcohol having lower volatility, for example, 1,2-propanediol or glycerol, is added to the ethanol in lesser amounts.

In a further exemplary embodiment, an agent as contemplated herein contains, based on its total weight, 1,2-propanediol and/or glycerol in a total amount of from about 0.1 to about 7.0% by weight, for example from about 0.5 to about 5.5% by weight, for example from about 1.0 to about 3.5% by weight and for example from about 1.5% by weight to about 2.5% by weight.

### Water Content

The water content of the agent can also influence the deposition of the color pigments (c) on the keratin materials and their film formation. If the water content is too high, there is a risk that the product will not dry sufficiently quickly. In particular, when the agents are adjusted to a lower viscosity (for example, because they are to be sprayed), the color result may be more uneven. In this case, an abrasion may already occur even before complete drying of the applied dyeings.

In the context of the work leading to this present disclosure, it has been found that a sufficiently rapid drying and, as a result, uniform film formation can be ensured in particular when the water content of the agent is set low.

In this context, a water content of between from about 0 and about 10% by weight, for example between from about 0 and about 8% by weight, for example between from about 0 and about 6% by weight and for example between from about 0 and about 3% by weight, has been found to be suitable. In this case, the water content specified in % by weight refers to the amount of water which is present in the total weight of the agent.

In a further exemplary embodiment, an agent as contemplated herein for changing the color of the keratinic materials has, based on its total weight, a water content of between from about 0 and about 8% by weight, for example between from about 0 and about 6% by weight, especially for example between from about 0 and about 3% by weight.

(b) Clay Minerals which are Modified with Quaternary Ammonium Compounds

The agents as contemplated herein contain at least one clay mineral modified with quaternary ammonium compounds as a second constituent (b) essential to the present disclosure. Here, the work leading to this present disclosure has shown that the clay minerals (b) modified with quaternary ammonium compounds are able to suspend the color pigments (c) in the alcoholic carrier medium (a) and stabilize them such that even after long transport and storage periods, agglomeration or conglomeration of the color pigments is effectively prevented at the bottom of the application vessel.

Clay minerals within the meaning of the present disclosure are understood to mean minerals which belong to the group of phyllosilicates (sheet silicates or leaf silicates) and band silicates. Clay minerals usually occur in the form of small, mostly flaky crystals (Ø<2 μm).

The clay minerals of group (b) modified with quaternary ammonium compounds are either colorless or white and are used in the form of colorless or white powders or gels. The modified clay minerals of group (b) are thus not suitable for achieving a color effect on the keratin materials, but serve as lipophilic thickening agents for suspending the actual color pigments (c). The modified clay minerals of group (b) are therefore explicitly excluded from the group of color pigments (c).

The silicate structure of the clay minerals is exemplified by sequences of tetrahedral layers and octahedral layers. Clay minerals having the sequence of one tetrahedral layer and one octahedral layer each are referred to as two-layer clay minerals or also 1:1 minerals. An example of a 1:1 clay mineral as contemplated herein is kaolinite. For kaolinite, the averaged composition Al₂[(OH)₄/Si₂O₅] or Al₂O₃.2SiO₂.2H₂O is is specified.

Clay minerals having formations of 1 octahedron layer and two tetrahedral layers are called three-layer minerals, or also 2:1 minerals. These include, for example, illite, the smectites, glauconite and vermiculite.

Illite is a mica-like three-layered clay mineral with the average composition K_{0.7}M_{0.1}⁺(Al,Fe³⁺)_{1.7}(Mg,Fe²⁺)_{0.3}[Si_{3.5}Al_{0.5}O₁₀(OH)₂].

Smectites belong to the phyllosilicates and are three-layered clay minerals (2:1 layered silicates), in which a central layer of octahedrally coordinated cations is surrounded sandwich-like by 2 layers of [(Si,Al)O₄] tetrahedra. Numerous substitutions take place in the octahedral layer; they can contain, besides mostly predominant Al³⁺ (montmorillonite), Mg²⁺ (saponite) or Fe³⁺ (nontronite), else cations such as Zn²⁺ (sauconite), Ni²⁺ (nickel S.) and Li⁺ (hectorite); in the tetrahedral layer, silicon can be replaced in part by Al³⁺ (for example, in beidellite) and also Fe³⁺ (in the case of nontronite). These substitutions result in an imbalance in the charge balance, which is compensated between the strata by exchangeable cations, usually sodium, calcium and potassium, also magnesium ions.

Belonging to the clay minerals, glauconite is an intense green, monoclinic hydrous potassium-iron-aluminum silicate having from about 2 to about 15% K₂O and a high Fe³⁺/Fe³⁺ ratio.

Vermiculite is one of the three-layered (2:1−) Phyllosilicates clay minerals; about (Mg,Al,Fe)₃[(Si,Al)₄O₁₀(OH)₂]Mgₓ(H₂O)ₙ, with 0.9>x>0.6. The silicon in the tetrahedral layers may be partly replaced by Al and Fe³⁺, the magnesium in the octahedron layers may be replaced in part by Al, Fe³⁺, secondarily also Fe²⁺; further may contain V, Ti, Ni, Cr, Zn and Mn as trace elements. The neg. charge x of the three-layer strata of 0.9>x>0.6 per formula unit is compensated by cations incorporated between the layers, easily interchangeable with each other and with Rb, Cs, Li, NH₄ and with organic compounds, especially Mg, but also Ca, Na and K. Oxides of Fe²⁺ to Fe³⁺ on the octahedral sites leads to a reduction of the interlayer charge. The layer charge can also be reduced directly by incorporating protons into structural OH groups.

Sepiolite (known as meerschaum, chemical formula Mg₄Si₆O₁₅(OH)_{2X}6H₂O) is a clay mineral having fibrous structure.

Palygorskite is a magnesium-rich, three-layer clay mineral having fibrous structure belonging to the phyllosilicates, containing more aluminum and less magnesium with respect to the similar sepiolite. The fibrous structure in palygorskite (known as mountain leather; chemical formula (Mg, Al)₂Si₄O₁₀(OH)ₓ4H₂O) is formed by papyrus-like or fibrous magnesium silicates, which are counted among the layer silicates. However, they differ from other layer silicates due to the lack of continuous octahedral layers.

Montmorillonite, for which the empirical formula Al₂[(OH)₂/Si₄O₁₀].nH₂O or Al₂O₃.4SiO₂.H₂O.nH₂O is specified, belongs to the clay minerals from the smectite group. Montmorillonite crystallizes monoclinic pseudohexagonal.

Saponite is a swellable, monoclinic clay mineral belonging to the smectites in which the octahedral layers in the structure are almost exclusively occupied by Mg, Fe²⁺, and Fe³⁺ ions. A neg. charge of x≤0.6 per O₁₀(OH)₂ in the tetrahedral layers is compensated by cations between the strata. Interlamellar pores are formed by incorporation of columns of polyoxo cations, e.g., Al₁₃⁷⁺ {via polyhydroxo cations [Al₁₃O₄(OH)₂₄(H₂O)₁₂]⁷⁺} (so-called pillared S.).

Hectorites belong to the smectites and have the approximate chemical formula M+0.3(Mg2.7Li0.3)[Si4O10(OH)2], wherein M+ is usually Na+.

Bentonites are clays and rocks that contain smectites, especially montmorillonite, as main minerals. The “raw” bentonites are either calcium bentonites (in Great Britain referred to as fuller earths) or sodium bentonites (also: Wyoming bentonites).

In these clay minerals, the strata can be shifted relative to one another relatively easily and thus yield the leaf-like structure for many clay minerals. Ions, water molecules and other substances (such as, for example, quaternary ammonium compounds) can incorporate between the strata, which can lead to an expansion of the layer spacings (swelling).

Synthetically produced smectites are also suitable as contemplated herein.

Clay minerals which are suitable for modification with quaternary ammonium compounds may be selected from montmorillonite, sepiolite, palygorskite, vermiculite, glauconite, hectorite, saponite, beidellite, nontronite, sauconite, bentonite and stevensite.

Exemplary clay minerals in the sense of the present disclosure may be selected from montmorillonite and sepiolite.

In an exemplary embodiment, an agent as contemplated herein
(b) contains at least one clay mineral modified with quaternary ammonium compounds, wherein the clay mineral is selected from the group of montmorillonite, sepiolite, hectorite, palygorskite, vermiculite, glauconite, saponite, beidellite, nontronite, sauconite, bentonite and stevensite, for example selected from montmorillonite and sepiolite.

The clay minerals are each treated or modified with quaternary ammonium compounds. Suitable quaternary ammonium compounds for the treatment of clay minerals and methods for the treatment of clay minerals with such compounds are known.

Suitable quaternary ammonium compounds are for example selected from quaternary alkylammonium salts of the following formula (I)

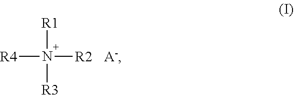

in which
R1 to R4 stand for the same or different, linear or branched, saturated or unsaturated
C1-C22 alkyl groups, or for benzyl radicals which may be substituted by one or more alkyl groups, and
A⁻ stands for a chloride, bromide, methylsulfate, nitrate, hydroxide, acetate and/or a phosphate anion.

At least one of the radicals R1 to R4 for example stands for a linear C1-C4 alkyl group, for example a methyl group, or a benzyl radical.

A⁻ is for example a chloride, bromide or methylsulfate ion.

Advantageous modified clay minerals in the context of the present disclosure are obtainable, for example, by converting clay minerals with Quaternium-18. Quaternium-18 is a mixture of quaternary ammonium chloride salts which are described by the following structural formula:

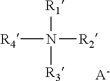

In Quatenernium-18, R1′ and R2′ both stand for methyl groups, and R3′ and R4′ independently of one another stand for a linear or branched, saturated or unsaturated C12-C20 alkyl group.

In the context of the embodiment, exemplary are, for example, stearalkonium hectorite, a reaction product of hectorite and stearalkonium chloride (benzyldimethylstearylammonium chloride), and Quaternium-18 hectorite, a reaction product of hectorite and Quaternium-18, which, for example, are available under the trade names Bentone 27 and Bentone 38 from Nordmann & Rassmann.

Quaternium-90 bentonite, a reaction product of bentonite and Quaternium-90, which is obtainable from Süd-Chemie under the trade name Tixogel VP-V, is also exemplary as contemplated herein.

Also exemplary is stearalkonium hectorite, which is commercially available under the name Bentone Gel IPM V in the form of a dispersion in isopropyl myristate and with an addition of propylene carbonate from the company Elementis Specialties.

Examples of exemplary quaternary ammonium compounds with which the clay minerals can be modified are dimethyl di(hydrogenated tallow) ammonium chloride, methylbenzyl di(hydrogenated tallow) ammonium chloride, dimethylbenzyl hydrogenated tallow ammonium chloride, dimethyl hydrogenated tallow-2-ethylhexylammonium chloride, cetyltrimethylammonium chloride, stearyltrimethylammonium chloride, behenyltrimethylammonium chloride and mixtures of these salts. The clay mineral mixture is for example treated with from about 5 to about 80 milliequivalents (meq.) of the quaternary ammonium compound (per 100 g of the clay mineral mixture).

As contemplated herein, the modification of the clay mineral with the quaternary ammonium compound is understood to mean the reaction between the quaternary ammonium compound and the clay mineral, which takes place by joint suspension of the previously pulverized clay mineral and quaternary ammonium compound in aqueous solution. Alternatively, the reaction between the quaternary ammonium compound and the clay mineral may also be described as the formation of ionic bonds. The clay minerals swell in the joint suspension of the clay mineral and the quaternary ammonium compound in water, since the water molecules diffuse into or between the previously described layers of the clay mineral. Together with the water, the quaternary ammonium compounds are also deposited (possibly by ion exchange) between these layers and in this way form the modified clay mineral.

In an exemplary embodiment, the clay minerals modified with quaternary ammonium compounds are prepared by pulverizing the corresponding clay mineral (for example, sepiolite, montmorillonite and/or hectorite), slurrying in water and filtering to remove sand and other contaminants. The slurry of each clay mineral is further diluted with water (from about 1 to about 6% solids) and passed into a mill with high shear (for example, a Manton-Gaulin mill) to achieve homogenization. The slurry is optionally passed through the mill with high shear several times. Such a method is described, for example, in U.S. Pat. No. 5,160,454, which is incorporated herein by reference. If only one clay mineral is to be modified with an ammonium group, then the quaternary ammonium compound is added to the clay mineral. If a mixture of clay minerals is to be modified with an ammonium group, for example, the respective clay minerals (for example, sepiolite and montmorillonite) can be first mixed, then the quaternary ammonium compound can be added. After dewatering, drying and, if appropriate, pulverizing of the mixtures, a modified clay mineral (or a mixture of the modified clay minerals) suitable as contemplated herein can be obtained.

Furthermore, it has been found that the color pigments (c) of the colorant can be kept in suspension finely divided over long periods of time, in particular when a mixture of clay minerals is used in the agent as contemplated herein, wherein these clay minerals are each modified with quaternary ammonium compounds.

It could be found that when using mixtures of clay minerals, which are each modified with quaternary ammonium compounds, a good improvement in storage stability could be produced. If at least two modified clay minerals were used in the agent as contemplated herein, on the one hand a good applicability was enabled, but on the other hand, especially the color pigments (c) could be suspended in suspension for a long time.

As contemplated herein, well-suited clay mineral mixtures modified with quaternary ammonium compounds are commercially available, for example, from Southern Clay Products under the trade name Garamite®. Exemplary clay mineral mixtures are Garamite® 7303, Garamite® 7305 and in particular Garamite® 7308 XR. Garamite 7308 XR consists essentially of a mixture of QUATERNIUM-90 SEPIOLITE and QUATERNIUM-90 MONTMORILLONITE.

Exemplary mixtures of modified clay minerals for the purposes of the present disclosure include
i) at least one clay mineral selected from sepiolite and/or palygorskite, and
ii) at least one clay mineral of the smectite type.

In an exemplary embodiment, an agent as contemplated herein
(bi) contains at least one first clay mineral modified with quaternary ammonium compounds, wherein the clay mineral is selected from sepiolite and/or palygorskite, and
(bii) contains at least one second clay mineral modified with quaternary ammonium compounds, wherein the clay mineral is a clay mineral of the smectite type, for example montmorillonite.

Clay minerals of type i) usually have a rod-like structure, while clay minerals from type ii) usually have a flaky structure. By mixing the clay mineral types i) and ii), nonuniform structures are achieved which allow the formation of spaces in which color pigments (c) can be excellently suspended. It is furthermore advantageous that agglomeration of the clay mineral mixture by the non-uniform structures is prevented or minimized, whereby the storage stability of the compositions as contemplated herein can be further increased. The modification of the clay mineral mixture with quaternary ammonium compounds produces repulsive, steric barriers that also help to disperse the clay mineral mixture.

The clay mineral mixture sepiolite is for example used as clay mineral i). The clay mineral mixture montmorillonite is for example used as clay mineral ii).

In an exemplary embodiment, an agent as contemplated herein
(bi) contains a first clay mineral modified with quaternary ammonium compound, wherein the clay mineral is sepiolite, and
(bii) contains a second clay mineral modified with quaternary ammonium compound, wherein the clay mineral is montmorillonite.

Furthermore, good results with regard to the object as contemplated herein were obtained when, in addition, a third clay mineral modified with a quaternary ammonium compound was used in the agents as contemplated herein.

In an exemplary embodiment, an agent as contemplated herein
(bi) contains a first clay mineral modified with quaternary ammonium compound, wherein the clay mineral is sepiolite, and
(bii) contains a second clay mineral modified with quaternary ammonium compound, wherein the clay mineral is montmorillonite, and
(biii) contains a third clay mineral modified with a quaternary ammonium compound, wherein the clay mineral is hectorite.

The best results were obtained from colorants which contained the combination of the raw materials Garamite® 7308 XR (mixture of QUATERNIUM-90 SEPIOLITE and QUATERNIUM-90 MONTMORILLONITE) and Bentone Gel IPM V (stearalkonium hectorite with the addition of propylene carbonate in isopropyl myristate).

It has been found that by using the previously described modified clay minerals, a much higher improvement in storage stability could be achieved compared to other organic thickeners.

The clay mineral(s) modified with quaternary ammonium compounds may be used in the agents as contemplated herein, based on the total weight of the agents, in a total amount of from about 0.1 to about 10.0% by weight, for example from about 0.5 to about 6.5% by weight, especially for example from about 1.0 to about 5.5% by weight and for example from about 1.5 to about 3.5% by weight.

In an exemplary embodiment, an agent as contemplated herein, based on its total weight,
(b) contains one or more clay minerals modified with quaternary ammonium compounds in a total amount of from about 0.1 to about 10.0% by weight, for example from about 0.5 to about 6.5% by weight, especially for example from about 1.0 to about 5.5% by weight and for example from about 1.5 to about 3.5% by weight.

### Color Pigments (c)

The agents as contemplated herein contain at least one color pigment as a third essential constituent (c). Color pigments in the context of the present disclosure are understood to mean coloring compounds which have a solubility of less than about 0.1 g/1 in water at about 20° C. The water solubility can be determined, for example, by the method described below: about 0.1 g of the color pigment is weighed in a beaker. A stir bar is added. It is then filled up to about 11 with distilled water (about 20° C.). It is stirred for an hour. If undissolved constituents of the color pigment are still visible in the mixture after this period, the solubility of the color pigment is below about 0.1 g/l.

A temporary dyeing of the keratinic material should take place with the cosmetic agents as contemplated herein. An example is when “metallic” effects are to be achieved. Therefore, the white pigments do not fall under the definition of the color pigment. White pigments are achromatic inorganic pigments having a high refractive index (usually greater than about 1.8), which are usually produced synthetically and especially for the production of optical whiteness in coating agents or as a filler, for example, in plastics. White pigments such as titanium dioxide or zinc dioxide are therefore explicitly excluded from the definition of the color pigment. The color pigments of group (c) are used for coloring, but do not remain for a long time in suspension in alcoholic solution; they therefore show no suitability as a suspending agent. The color pigments (c) therefore do not belong explicitly to the group of modified clay minerals (b).

In the agents, the color pigments (c) are present in the form of small undissolved particles which do not diffuse into the keratinic surface, but which attach to the outside of the keratinic surface in the form of a film during the application of the colorant.

Suitable color pigments may be of organic and/or inorganic origin. Exemplary color pigments are selected from synthetic or natural inorganic color pigments. Inorganic color pigments of natural origin can be prepared, for example, from chalk, ocher, umber, green earth, baked Terra di Siena or graphite. Furthermore, black pigments such as iron oxide black, colored pigments such as ultramarine or iron oxide red and fluorescent or phosphorescent pigments be used as an inorganic color pigment.

Exemplary embodiments of the first subject of the present disclosure are therefore exemplified in that the cosmetic agent contains as color pigment (c) at least one inorganic color pigment which is selected from (i) colored metal oxides, (ii) metal hydroxides, (iii) metal oxide hydrates, (iv) silicates, (v) metal sulfides, (vi) complex metal cyanides, (vii) metal sulfates, (viii) bronze pigments and/or (ix) mica-based colored color pigments which are coated with at least one metal oxide and/or metal oxychloride, and (x) their mixtures.

Suitable are colored metal oxides, hydroxides and oxide hydrates, mixed phase pigments, sulfur-containing silicates, silicates, metal sulfides, complex metal cyanides, metal sulfates, chromates and/or molybdates. Exemplary color pigments are black iron oxide (CI 77499), yellow iron oxide (CI 77492), red and brown iron oxide (CI 77491), manganese violet (CI 77742), ultramarines (sodium aluminum sulfosilicates, CI 77007, color pigment Blue 29), chromium oxide hydrate (CI77289), iron blue (ferric ferrocyanide, CI77510) and/or carmine (cochineal).

In an exemplary embodiment, an agent as contemplated herein contains as color pigment (c) at least one inorganic color pigment which is selected from colored metal oxides, metal hydroxides, metal oxide hydrates, silicates, metal sulfides, complex metal cyanides, metal sulfates, bronze pigments and/or of colored pigments based on mica, which are coated with at least one metal oxide and/or one metal oxychloride.

Color pigments (c) exemplary as contemplated herein are colored pearlescent pigments. These are usually based on mica and may be coated with one or more metal oxides. Mica belongs to the layer silicates. The most important representatives of these silicates are muscovite, phlogopite, paragonite, biotite, lepidolite and margarite. To prepare the pearlescent pigments in conjunction with metal oxides, the mica, predominantly muscovite or phlogopite, is coated with a metal oxide.

As an alternative to natural mica, it is also possible to use synthetic mica coated with one or more metal oxides as a pearlescent pigment. Exemplary pearlescent pigments are based on natural or synthetic mica and are coated with one or more of the abovementioned metal oxides. The color of the respective color pigments can be varied by varying the layer thickness of the metal oxide(s).

It is therefore exemplary as contemplated herein when the at least one color pigment (c) is a mica-based colored pigment which contains one or more metal oxides from the group including of titanium dioxide (CI 77891), black iron oxide (CI 77499), yellow iron oxide (CI 77492), red and/or brown iron oxide (CI 77491, CI 77499), manganese violet (CI 77742), ultramarines (sodium aluminum sulfosilicates, CI 77007, color pigment Blue 29), chromium oxide hydrate (CI 77289), chromium oxide (CI 77288) and/or iron blue (ferric ferrocyanide, CI 77510).

In an exemplary embodiment, an agent as contemplated herein is exemplified in that the at least one color pigment (c) is a colored pigment based on mica which is coated with one or more metal oxides from the group of titanium dioxide (CI 77891), black iron oxide (CI 77499), yellow iron oxide (CI 77492), red and/or brown iron oxide (CI 77491, CI 77499), manganese violet (CI 77742), ultramarines (sodium aluminum sulfosilicates, CI 77007, color pigment Blue 29), chromium oxide hydrate (CI 77289), chromium oxide (CI 77288) and/or iron blue (ferric ferrocyanide, CI 77510).

Examples of suitable color pigments (c) are commercially available, for example, under the trade names Rona®, Colorona®, Xirona®, Dichrona® and Timiron® from Merck, Ariabel® and Unipure® from Sensient, Prestige® from Eckart Cosmetic Colors and Sunshine® from Sunstar.

Exemplary color pigments with the trade name Colorona® are, for example:
Colorona Copper, Merck, MICA, CI 77491 (IRON OXIDES)
Colorona Passion Orange, Merck, Mica, CI 77491 (Iron Oxides), Alumina
Colorona Patina Silver, Merck, MICA, CI 77499 (IRON OXIDES), CI 77891 (TITANIUM DIOXIDE)
Colorona RY, Merck, CI 77891 (TITANIUM DIOXIDE), MICA, CI 75470 (CARMINE)
Colorona Oriental Beige, Merck, MICA, CI 77891 (TITANIUM DIOXIDE), CI 77491 (IRON OXIDES)
Colorona Dark Blue, Merck, MICA, TITANIUM DIOXIDE, FERRIC FERROCYANIDE
Colorona Chameleon, Merck, CI 77491 (IRON OXIDES), MICA
Colorona Aborigine Amber, Merck, MICA, CI 77499 (IRON OXIDES), CI 77891 (TITANIUM DIOXIDE)
Colorona Blackstar Blue, Merck, CI 77499 (IRON OXIDES), MICA
Colorona Patagonian Purple, Merck, MICA, CI 77491 (IRON OXIDES), CI 77891 (TITANIUM DIOXIDE), CI 77510 (FERRIC FERROCYANIDE)
Colorona Red Brown, Merck, MICA, CI 77491 (IRON OXIDES), CI 77891 (TITANIUM DIOXIDE)
Colorona Russet, Merck, CI 77491 (TITANIUM DIOXIDE), MICA, CI 77891 (IRON OXIDES)
Colorona Imperial Red, Merck, MICA, TITANIUM DIOXIDE (CI 77891), D&C RED NO. 30 (CI 73360)
Colorona Majestic Green, Merck, CI 77891 (TITANIUM DIOXIDE), MICA, CI 77288 (CHROMIUM OXIDE GREENS)
Colorona Light Blue, Merck, MICA, TITANIUM DIOXIDE (CI 77891), FERRIC FERROCYANIDE (CI 77510)
Colorona Red Gold, Merck, MICA, CI 77891 (TITANIUM DIOXIDE), CI 77491 (IRON OXIDES)
Colorona Gold Plus MP 25, Merck, MICA, TITANIUM DIOXIDE (CI 77891), IRON OXIDES (CI 77491)
Colorona Carmine Red, Merck, MICA, TITANIUM DIOXIDE, CARMINE
Colorona Blackstar Green, Merck, MICA, CI 77499 (IRON OXIDES)
Colorona Bordeaux, Merck, MICA, CI 77491 (IRON OXIDES)
Colorona Bronze, Merck, MICA, CI 77491 (IRON OXIDES)
Colorona Bronze Fine, Merck, MICA, CI 77491 (IRON OXIDES)
Colorona Fine Gold MP 20, Merck, MICA, CI 77891 (TITANIUM DIOXIDE), CI 77491 (IRON OXIDES)
Colorona Sienna Fine, Merck, CI 77491 (IRON OXIDES), MICA
Colorona Sienna, Merck, MICA, CI 77491 (IRON OXIDES)
Colorona Precious Gold, Merck, Mica, CI 77891 (Titanium dioxide), Silica, CI 77491(Iron oxides), Tin oxide
Colorona Sun Gold Sparkle MP 29, Merck, MICA, TITANIUM DIOXIDE, IRON OXIDES, MICA, CI 77891, CI 77491 (EU)
Colorona Mica Black, Merck, CI 77499 (Iron oxides), Mica, CI 77891 (Titanium dioxide)
Colorona Bright Gold, Merck, Mica, CI 77891 (Titanium dioxide), CI 77491(Iron oxides)
Colorona Blackstar Gold, Merck, MICA, CI 77499 (IRON OXIDES)

Further exemplary color pigment with the trade name Xirona® are, for example:
Xirona Golden Sky, Merck, Silica, CI 77891 (Titanium Dioxide), Tin Oxide
Xirona Caribbean Blue, Merck, Mica, CI 77891 (Titanium Dioxide), Silica, Tin Oxide
Xirona Kiwi Rose, Merck, Silica, CI 77891 (Titanium Dioxide), Tin Oxide
Xirona Magic Mauve, Merck, Silica, CI 77891 (Titanium Dioxide), Tin Oxide.

In addition, exemplary color pigment with the trade name Unipure® are, for example:
Unipure Red LC 381 EM, Sensient CI 77491 (Iron Oxides), Silica
Unipure Black LC 989 EM, Sensient, CI 77499 (Iron Oxides), Silica
Unipure Yellow LC 182 EM, Sensient, CI 77492 (Iron Oxides), Silica

Due to their excellent light, weather and/or temperature resistance, the use of the abovementioned inorganic color pigments (c) is exemplary in the agent as contemplated herein. Furthermore, it is exemplary when the color pigments used have a certain particle size. On the one hand, this particle size leads to a uniform distribution of the color pigments in the polymer film formed and, on the other hand, avoids a rough hair or skin feeling after the application of the cosmetic agent. It is therefore advantageous as contemplated herein when the at least one color pigment has an average particle size D₅₀ of from about 1.0 to about 50 μm, for example from about 5.0 to about 45 μm, for example from about 10 to about 40 μm, in particular from about 14 to about 30 μm. The average particle size D₅₀ can be determined, for example, using dynamic light scattering (DLS).

Depending on which color change on the surface of the keratinic material is desired, the at least one color pigment (c) can be used in different amounts. The more color pigment used, the higher the extent of color change generally. At the same time, the higher the content of color pigments (c) in the agent, the more difficult it becomes to prepare the agents in a storage-stable form. In this context, the work leading to this present disclosure has shown that the undyed and well suspendable modified clay minerals (b) show a disproportionately good effect, especially with high amounts of color pigments (c) used. Good stabilization and suspension over long storage periods could be achieved, especially with pigment amounts of up to about 25% by weight (based on the total weight of the agent).

In an exemplary embodiment, an agent as contemplated herein, based on its total weight,

(c) contains one or more color pigments in a total amount of from about 1.0 to about 25.0% by weight, for example from about 5.0 to about 20.0% by weight, for example from about 7.0 to about 18.0% by weight, in particular from about 8.5 to about 15.5% by weight.

### Oils

It is advantageous to add at least one oil to the agents as contemplated herein to improve the water resistance. In the application of the agent, the oil, together with the color pigments (c), forms a film on the surface of the keratin material; the hydrophobic character of this film helps to prevent premature rinsing by rain and perspiration.

Especially exemplary is the use of liquid oils. As contemplated herein, a liquid oil is to be understood to mean a flowable substance at room temperature (about 25° C. and about 1013 mbar) which is miscible under normal conditions with bidistilled water to less than about 1% by weight.

According to an exemplary embodiment, the agent as contemplated herein contains as oil at least one ester of a C₈-C₂₀ fatty acid and a C₁-C₂₀ alcohol. The ester may be selected from esters which are formed from at least one linear or branched, saturated or unsaturated monocarboxylic acid having from about 8 to about 20 carbon atoms, which may optionally contain one or more hydroxyl groups, and at least one linear or branched, saturated or unsaturated alcohol having from about 1 to about 20 carbon atoms.

Suitable esters are selected from isopropyl myristate, isopropyl laurate, isopropyl palmitate, isopropyl stearate, 2-ethylhexyl laurate, 2-ethylhexyl myristate, 2-ethylhexyl palmitate, 2-ethylhexyl cocoate, 2-ethylhexyl stearate, 2-ethylhexyl isostearate, hexyldecyl laurate, hexyldecyl stearate, isooctyl stearate, isononyl isononanoate, isononyl stearate, isotridecylnonanoate, 2-octyl dodecyl palmitate, isocetyl stearate and/or mixtures of these esters.

In particular, isopropyl myristate, isopropyl laurate, isopropyl palmitate and isopropyl stearate are well distributed on the keratin materials and together with the color pigments (c) form stable, hydrophobic films.

In an exemplary embodiment, an agent as contemplated herein further contains one or more oils, for example selected from the group including of isopropyl myristate, isopropyl laurate, isopropyl palmitate, isopropyl stearate, 2-ethylhexyl laurate, 2-ethylhexyl myristate, 2-ethylhexyl palmitate, 2-ethylhexyl cocoate, 2-ethylhexyl stearate, 2-ethylhexyl isostearate, hexyldecyl laurate, hexyldecyl stearate, isooctyl stearate, isononyl isononanoate, isononyl stearate, isotridecylnonanoate, 2-octyldodecyl palmitate and isocetyl stearate, for example selected from isopropyl myristate, isopropyl laurate, isopropyl palmitate and isopropyl stearate.

Such cosmetic agents have a high resistance to external environmental influences, such as rain and perspiration, and can still be removed without residue by the use of surfactant-containing cleaning agents. When applied to keratinic fibers, in particular hair, an excellent degree of hold and a high moisture resistance is also achieved. The high moisture resistance avoids a sticking of the hair when exposed to moisture. When applied to the skin, these agents have good cosmetic properties and do not lead to dry or oily skin.

Stable agents having good performance properties could be obtained when modified clay minerals (b) were used for the suspension of the color pigments (c), which clay minerals were already present in the form of a predispersion in one of the aforementioned oils. When using appropriate predispersions, the desired viscosity of the agent could be set precisely and co-sintering of the color pigments (b) could be prevented effectively.

A suitable dispersion is, for example, the raw material sold by Elementis Specialties under the trade name Bentone Gel IPM V, from about 5 to about 20% by weight of stearalkonium hectorite and from about 1 to about 10% by weight of propylene carbonate dispersed in isopropyl myristate (ad about 100% by weight).

Also suitable is the use of other oils such as silicone oils. A suitable raw material in this context is Bentone Gel 1002 V from Elementis Specialties, which contains disteardimonium hectorite and propylene carbonate dispersed in cyclopentasiloxane.

### Other Polymers

A finely divided suspension or dispersion can be achieved through the joint use of the modified clay minerals (b) and the color pigments (c) in the alcoholic carrier (a), which prevents the color pigments from sinking in the long term. A layer lattice appears to form in the suspension of the two constituents (b) and (c) in (a), which prevents agglomeration of the color pigments (c).

Further work has shown that this layer lattice can be disturbed by the use of additional polymers. In particular, the layered lattice may collapse when certain anionic polymers are used together with the modified clay minerals (b). A prevention of the sedimentation of the color pigments (c) over long periods is then no longer possible in this case.

For this reason, it may turn out to be exemplary when the total amount of anionic polymers contained in the agent as contemplated herein, based on the total weight of the agent, is below about 2.5% by weight, for example below about 1.0% by weight, for example below about 0.5% by weight and for example below about 0.1% by weight.

In an exemplary embodiment, an agent as contemplated herein is exemplified in that the total amount of the anionic polymers contained in the agent as contemplated herein, based on the total weight of the agent, is below about 2.5% by weight, for example below about 1.0% by weight, for example below about 0.5% by weight and for example below about 0.1% by weight.

As contemplated herein, an anionic polymer is understood to mean an organic polymer which carries structural units with anionic groups in a protic solvent, in particular in a water-containing environment, under standard conditions. To retain electroneutrality, these anionic groups are neutralized by stoichiometric equivalents of counterions, such as alkali metal cations, alkaline earth metal cations, or ammonium ions (NH₄⁺). The group of anionic groups present in the polymer includes carboxyl groups, sulfonic acid groups and phosphonic acid groups, in particular carboxy and sulfonic acid groups. When the polymer is used, the carboxylic acid and sulfonic acid groups may also initially be present in protonated (uncharged) form and then converted into the corresponding anionic form upon contact with the aqueous environment by elimination of a proton.

Anionic polymers are, for example
(i) polymers of acrylic acid and/or methacrylic acid,
(ii) polymers of 2-acrylamido-2-methyl-1-propanesulfonic acid,
(iii) anionic polysaccharides,
(iv) polymers of itaconic acid and/or crotonic acid and/or
(v) polymers of maleic anhydride.

In an exemplary embodiment, an agent as contemplated herein is exemplified in that the total amount of anionic polymers contained in the agent of the present disclosure from the group of
(i) polymers of acrylic acid and methacrylic acid, and
(ii) polymers of 2-acrylamido-2-methyl-1-propanesulfonic acid and
(iii) anionic polysaccharides and
(iv) polymers of itaconic acid and crotonic acid and
(v) polymers of maleic anhydride,
   based on the total weight of the agent, is below about 2.5% by weight, for example below about 1.0% by weight, for example below about 0.5% by weight and for example below about 0.1% by weight.

The homopolymers and copolymers of type (i) are exemplified in that they contain at least one structural unit of the formula (P-I),

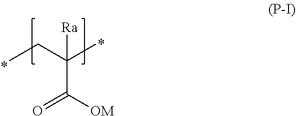

wherein
Ra stands for a hydrogen atom or a methyl group and
M stands for a hydrogen atom or for sodium, potassium, ½ magnesium or ½ calcium.

A homopolymer of (i) is, for example, polyacrylic acid.

Also sold under the name Simulgel®EG, as a compound with isohexadecane and polysorbate-80, is an anionic polymer sodium acrylate/acryloyldimethyl taurate copolymer copolymers (INCI name: SODIUM ACRYLATE/SODIUM ACRYLOYLDIMETHYL TAURATE COPOLYMER, ISOHEXADECANE, POLYSORBATE 80).

At least one copolymer of acrylic acid and/or methacrylic acid may also be mentioned as an anionic polymer. The polymer known under the INCI name Acrylates/C10-30 Alkyl Acrylate Crosspolymer, available under the trade name Carbopol 1382 from Noveon, belongs to this group.

A further anionic polymer is the polymer known under the INCI name Acrylates/Steareth-20 Methacrylate Crosspolymer, which is sold, for example, under the trade name Aculyn® 88 from Rohm & Haas in the form of a from about 28 to about 30% by weight dispersion in water. Furthermore, polymers can be named according to the INCI nomenclature as Acrylates/Palmeth-25 Acrylate Copolymer or Acrylates/Palmeth-20 Acrylate Copolymer.

Examples of further anionic copolymers are copolymers of acrylic acid and/or methacrylic acid and their C₁-C₆ alkyl esters, as sold under the INCI declaration Acrylates Copolymers.

A commercial product of this group is, for example, Aculyx 33 from Rohm & Haas. Other representatives are also copolymers of acrylic acid and/or methacrylic acid, the C1-C6 alkyl esters of acrylic acid and/or methacrylic acid and the esters of an ethylenically unsaturated acid and an alkoxylated fatty alcohol. Ethylenically unsaturated acids are in particular acrylic acid, methacrylic acid and itaconic acid; suitable alkoxylated fatty alcohols are in particular steareth-20 or ceteth-20. Such copolymers are sold by Rohm & Haas under the tradename Aculyn® 22 (INCI Name: Acrylates/Steareth-20 Methacrylate Copolymer).

Further anionic polymers are, for example, a copolymer of (meth)acrylic acid and octylacrylamide, as are commercially available, for example, under the trade name Dermacryl 79 from Akzo Nobel.

Anionic polymers of type (ii) are homo- and co-polymers of 2-acrylamido-2-methyl-1-propanesulfonic acid. Polymers of type (ii) are exemplified in that they contain as a structural component at least one structural unit of the formula (P-II),

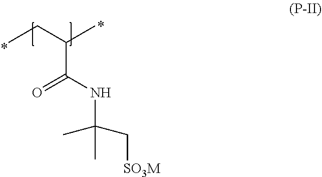

wherein
M stands for a hydrogen atom or for sodium, potassium, ½ magnesium or ½ calcium.

Polymers of this type have anionic sulfonic acid groupings which are incorporated into the polymer by polymerization of the monomer 2-methyl-2-[(1-oxo-2-propenyl)amino]-1-propanesulfonic acid (AMPS).

For example, the polymer known under the name Cosmedia HSP 1160 and the product commercially available under the name Rheothik®11-80 may be mentioned as homopolymers of 2-methyl-2[(1-oxo-2-propenyl)amino]-1-propanesulfonic acid.

Further anionic sulfonic acid polymers of this kind are copolymers of 2-methyl-2[(1-oxo-2-propenyl)amino]-1-propanesulfonic acid (AMPS) and sodium acrylate, which are available, for example, as a commercial product Simulgel® EG (INCI name: Sodium Acrylates/Sodium Acryloyldimethyl Taurate Copolymer, Isohexadecane, Polysorbate 80) from Seppic.

Anionic polymers from the group of anionic polysaccharides (iii) are, for example, xanthans, alginates, carboxyalkylcelluloses and hyaluronic acids.

Anionic polymers of group (iv) are homo- and co-polymers of itaconic acid and/or crotonic acid. Polymers of this type are exemplified in that they contain, as structural constituents, at least one unit of the formula (P-III) and/or of the formula (P-IV),

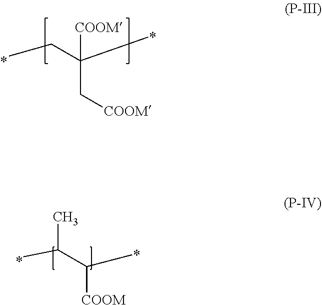

wherein
M, M′, M″ each independently of one other stands for a hydrogen atom or for sodium, potassium, ½ magnesium or ½ calcium.

A copolymer belonging to this class is, for example, the terpolymer which can be prepared by the copolymerization of vinyl chloride, vinyl acetate and itaconic acid, which terpolymer is also commercially available under the trade name Vinnol E 15/45 M from Wacker Polymer Systems.

Anionic polymers of group (v) are homo- and co-polymers of maleic anhydride. This group includes homopolymers and copolymers containing at least one structural component of the formula (P-V), which is formed by the polymerization and hydrolysis of the maleic anhydride monomer building blocks,

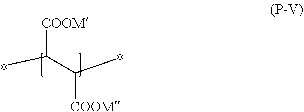

wherein
M′, M″ independently of each other stand for a hydrogen atom or for sodium, potassium, ½ magnesium or ½ calcium.

The copolymer of maleic anhydride and methyl vinyl ether may be mentioned in this context, for example. A maleic acid-methyl copolymer crosslinked with 1,9-decadiene is commercially available under the name Stabileze® QM.

As described above, with regard to the stable suspension of the color pigments (c), it has proven to be advantageous when all of these aforementioned anionic polymers are present in the agent in a total amount which is less than about 2.5% by weight, for example less than of about 1.0% by weight, for example less than about 0.5% by weight and for example less than about 0.1% by weight.

It is exemplary when the agents are substantially free of anionic polymers, by which is meant that their total amount, based on the total weight of the agent, is below about 0.1% by weight.

### Other Ingredients

The cosmetic agents of the present disclosure can be used for color change, in particular for the temporary changing of the color, of keratinic materials, in particular hair and skin. They can be made up in conventional forms for this purpose, for example, as a gel, spray, foam or wax. Preference is given to the preparation as a spray. Such sprays may be in the form of aerosols and non-aerosols and sprayed from containers known to one skilled in the art. If the agents are formulated as aerosols, at least one propellant is additionally present. Propellants which are suitable as contemplated herein are selected, for example, from N₂O, dimethyl ether, CO₂, air, alkanes having from about 3 to about 5 carbon atoms, such as propane, n-butane, isobutane, n-pentane and isopentane, and mixtures thereof. Very particular preference is given to using dimethyl ether as a propellant. However, the present disclosure expressly also includes the concomitant use of propellants of the type of chlorofluorohydrocarbons, but in particular of fluorocarbons. These propellants are for example present in a total amount of from about 30 to about 70% by weight, for example from about 35 to about 75% by weight, in particular from about 40 to about 60% by weight, based on the total weight of the cosmetic agent.

In addition to the components described above, the cosmetic agents of the present disclosure may contain further ingredients. The group of these other ingredients includes, in particular, cosmetically-effective auxiliaries and additives, such as surfactants, care substances, thickeners and pH stabilizers.

The agents as contemplated herein may additionally contain at least one nonionic surfactant. Suitable nonionic surfactants are alkyl polyglycosides and alkylene oxide addition products of fatty alcohols and fatty acids with in each case from about 2 to about 30 moles of ethylene oxide per mole of fatty alcohol or fatty acid. Preparations having good properties are also obtained when they contain, as nonionic surfactants, fatty acid esters of ethoxylated glycerol which have been converted with at least about 2 moles of ethylene oxide. The nonionic surfactants are used in a total amount of from about 0.1 to about 45% by weight, for example from about 1 to about 30% by weight and for example from about 1 to about 15% by weight, based on the total weight of the agent.

The agents as contemplated herein may additionally contain at least one cationic surfactant. Cationic surfactants are understood to mean surfactants, that is, surface-active compounds, each having one or more positive charges. Cationic surfactants contain only positive charges. Usually, these surfactants are made up of a hydrophobic part and a hydrophilic head group, wherein the hydrophobic part usually of a hydrocarbon skeleton (for example, including of one or two linear or branched alkyl chains), and the positive charge(s) are located in the hydrophilic head group. Examples of cationic surfactants are
quaternary ammonium compounds which, as hydrophobic radicals, may be able to carry one or two alkyl chains having a chain length of from about 8 to about 28 C atoms,
quaternary phosphonium salts substituted with one or more alkyl chains having a chain length of from about 8 to about 28 C atoms or
tertiary sulfonium salts.

Furthermore, the cationic charge can also be in the form of an onium structure component of a heterocyclic ring (for example, an imidazolium or a pyridinium ring). In addition to the functional unit which carries the cationic charge, the cationic surfactant may also contain further uncharged functional groups, as is the case, for example, with esterquats. The cationic surfactants are used in a total amount of from about 0.1 to about 45% by weight, for example from about 1 to about 30% by weight and for example from about 1 to about 15% by weight, based on the total weight of the agent.

The use of anionic surfactants has been found to be negative in terms of the abrasion resistance of the color pigments on the keratin materials and the storage stability of the agents. For this reason, it is exemplary to not use anionic surfactants in the agents as contemplated herein. Anionic surfactants are surface-active agents having exclusively anionic charges (neutralized by a corresponding counter cation). Examples of anionic surfactants are fatty acids, alkyl sulfates, alkyl ether sulfates and ether carboxylic acids having from about 12 to about 20 carbon atoms in the alkyl group and up to about 16 glycol ether groups in the molecule.

In a further exemplary embodiment, agents as contemplated herein are exemplified in that they contain anionic surfactants in a total amount of from about 0 to about 2.5% by weight, for example from about 0 to about 1.5% by weight, for example from about 0 to about 0.5% by weight, yet for example from about 0 to about 0.1% by weight, in particular of about 0% by weight, each based on the total weight of the cosmetic agent.

The agents as contemplated herein may furthermore contain at least one zwitterionic and/or amphoteric surfactant. Suitable zwitterionic surfactants are betaines, N-alkyl-N,N-dimethylammonium glycinates, N-acyl-aminopropyl-N,N-dimethylammonium glycinates, and 2-alkyl-3-carboxymethyl-3-hydroxyethyl-imidazolines. An exemplary zwitterionic surfactant is known by the INCI name Cocamidopropyl Betaine. Suitable amphoteric surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkyl sarcosines, 2-alkylaminopropionic acids and alkylaminoacetic acids. Exemplary amphoteric surfactants are N-cocoalkylaminopropionate, cocoacylaminoethylaminopropionate and C12-C18 acylsarcosine.

The amphoteric and/or zwitterionic surfactants are used in a total amount of from about 0.1 to about 45% by weight, for example from about 1 to about 30% by weight and for example from about 1 to about 15% by weight, based on the total weight of the agent.

An advantage of the agents as contemplated herein is that they can be formulated in a variety of forms. Very uniform color effects and water and rubbed dyeings can be achieved when applied via a sponge or a small brush. Likewise, however, it is also possible to formulate the agents as contemplated herein as a spray. In particular, the dyeings obtained by the spray application are exemplified by a very high uniformity and water resistance.

The agents as contemplated herein are adjusted to a specific viscosity, depending on the chosen application form. The adjustment of the desired viscosity is for example carried out via the modified clay minerals (b).

The agents as contemplated herein may contain one or more alkalizing agents to adjust the pH value. The alkalizing agents which can be used as contemplated herein for setting the desired pH values can be selected from the group which is formed of ammonia, alkanolamines, basic amino acids and inorganic alkalizing agents such as (earth) alkali metal hydroxides, (earth) alkali metal metasilicates, (earth) alkaline metal phosphates and (earth) alkali metal hydrogen phosphates. The agents as contemplated herein may contain one or more acids to adjust the pH value. Suitable acids are, for example, organic acids such as alpha-hydroxycarboxylic acids or inorganic acids.

Further optional ingredients are, for example, nonionic, fully synthetic polymers, such as polyvinyl alcohol or polyvinylpyrrolidinone. Suitable nonionic polymers are, for example:
vinylpyrrolidone/vinyl ester copolymers,
starch and its derivatives, in particular starch ethers,
shellac
polyvinylpyrrolidones.

Furthermore, the agents as contemplated herein may contain further active ingredients, auxiliaries and additives, for example, linear cationic polymers such as quaternized cellulose ethers, polysiloxanes with quaternary groups, with diethyl sulfate-quaternized dimethylamino-ethylmethacrylate-vinylpyrrolidinone copolymers, vinylpyrrolidinone-imidazolinium methochloride copolymers and quaternized polyvinyl alcohol; zwitterionic and amphoteric polymers (other than the polymers of the present disclosure); structurants such as glucose, maleic acid and lactic acid, hair conditioning compounds such as phospholipids, for example, lecithin and cephalins; perfume oils, dimethylisosorbide and cyclodextrins; active ingredients that improve fiber structure, in particular mono-, di- and oligosaccharides such as, glucose, galactose, fructose, fruit sugar and lactose; dyes for staining the agent; anti-dandruff agents such as Piroctone Olamine, Zinc Omadine and Climbazole; amino acids and oligopeptides; protein hydrolysates on animal and/or vegetable basis, and in the form of their fatty acid condensation products or optionally anionically or cationically modified derivatives; sunscreens and UV blockers; active ingredients such as panthenol, pantothenic acid, pantolactone, allantoin, pyrrolidinonecarboxylic acids and their salts, and bisabolol; polyphenols, in particular hydroxycinnamic acids, 6,7-dihydroxycoumarins, hydroxybenzoic acids, catechins, tannins, leucoanthocyanidins, anthocyanidins, flavanones, flavones and flavonols; ceramides or pseudoceramides; vitamins, provitamins and vitamin precursors; plant extracts; fats and waxes such as fatty alcohols, beeswax, montan wax and paraffins; swelling and penetrating substances such as glycerol, propylene glycol monoethyl ether, carbonates, hydrocarbonates, guanidines, ureas and primary, secondary and tertiary phosphates; opacifiers such as latex, styrene/PVP and styrene/acrylamide copolymers, and PEG-3-distearate; propellants such as propane-butane mixtures, N₂O, dimethyl ether, CO₂ and air.

The selection of these further substances will be made by one skilled in the art according to the desired properties of the agents. With regard to further optional components and the amounts of these components used, reference is expressly made to the relevant manuals known to one skilled in the art. The additional active ingredients and auxiliaries are for example used in the agents as contemplated herein in amounts of from about 0.0001 to about 25% by weight, in particular from about 0.0005 to about 15% by weight, based on the total weight of the cosmetic product.

### Method

When using the agent in the form of a pump spray or in the form of an aerosol spray, the user can spray the agents as contemplated herein directly on the dry hair or skin and in this way produce the desired temporary color change.

A second subject of the present disclosure is therefore a method for the temporary dyeing of keratinic materials, in particular of hair and/or the skin, wherein the method includes the following method steps:
a) providing an agent, as was disclosed in detail in the description of the first subject of the present disclosure, in a pump-spray container or an aerosol-spray container, and
b) applying, in particular spraying, the cosmetic agent provided in step a) to the keratinic materials, in particular to the hair and/or the skin.

With regard to further exemplary embodiments of the method as contemplated herein, in particular with regard to the cosmetic agent used there, mutatis mutandis applies to the agents of the present disclosure.

### EXAMPLES:

### 1. Formulations

The following compositions were prepared (all specifications in percent by weight, based on the total weight of the respective cosmetic agent):

| | V1 | V2 | E1 | E2 |
|---|---|---|---|---|
| Glycerin | 0.50 | 0.50 | 0.50 | 0.50 |
| Propylene carbonate | 0.90 | 0.90 | 0.90 | 0.90 |
| D-panthenol | 0.20 | 0.20 | 0.20 | 0.20 |
| Colorona Bronze, Merck, | 11.00 | 11.00 | 11.00 | 11.00 |
| MICA, CI 77491 (IRON | | | | |
| OXIDES) | | | | |
| Dermacryl 79 (Akzo Nobel, | 2.50 | — | — | — |
| Acrylates/Octylacrylamide | | | | |
| Copolymer) | | | | |
| Xanthan | — | 2.50 | — | — |
| Bentone Gel IPM V | — | — | — | 6.00 |
| (elementis, isopropyl | | | | |
| myristate, stearalkonium | | | | |
| hectorite, propylene | | | | |
| carbonate) | | | | |
| Garamite 7308 XR | — | — | 2.50 | 2.50 |
| (Quaternium-90 Sepiolite, | | | | |
| Quatenrium-90 | | | | |
| Montmorillonite) | | | | |
| Ethanol | ad 100 | ad 100 | ad 100 | ad 100 |

### 2. Test of Storage Stability

Each of the formulations V1, V2, E1 and E2 was filled into a transparent PE bottle (pump-spray container) and allowed to stand for 4 weeks. The bottles were not moved during the storage period of 4 weeks.

After 4 weeks, the color pigments in the bottles had sunk to the bottom to a greatly different extent and formed a layer on the bottom of the bottle.

An effort was now made to bring the color pigments back into suspension by shaking. The suspensibility of the dye pigments was evaluated and the spray pattern that was obtained after the 4 weeks storage period was evaluated.

| | V1 | V2 | E1 | E2 |
|---|---|---|---|---|
| Sedimen- | dense layer | dense layer | slight | slight |
| tation | at the | at the | subsidence, | subsidence, |
| | bottom of | bottom of | pigments | pigments |
| | the bottle | the bottle | remained | remained |
| | | | suspended | suspended |
| Shake for | pigment layer | pigment layer | suspension | suspension |
| 2 min | was not re- | was not re- | | |
| | suspendable | suspendable | | |
| | by shaking | by shaking | | |
| Spray | uneven, nozzle | uneven, nozzle | even bronze | even bronze |
| pattern | clogged after | clogged after | color | color |
| | some time | some time | | |
| Overall | unacceptable | unacceptable | good | good |
| evaluation | | | | |

### 3. Test for Water Resistance

Only the formulations which were considered to be at least acceptable in the overall storage stability evaluation were dyed.

The agents E1 and E2 were each applied to a strand of hair (Kerling 6-0, light brown). After drying, a uniform bronze-colored metallic dyeing was obtained in both cases. Subsequently, both strands were washed with water (30 seconds). There was an evaluation of whether the strand was completely decolorized within this period.

| | E1 | E2 |
|---|---|---|
| Wash the strand with | almost completely decolorized | hardly any loss |
| water for 30 s | | of color intensity |

The strand dyed with formulation E2 had better water resistance compared to the strand dyed with E1.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. An agent for changing the color of keratinic materials comprising:
(a) at least one aliphatic and/or aromatic alcohol having from 2 to about 8 carbon atoms,
(b) at least one clay mineral modified with quaternary ammonium compounds, and
(c) at least one color pigment.

2. The agent according to claim 1, wherein the agent comprises one or more alcohols (a) chosen from ethanol, isopropanol, n-propanol, butanol, n-pentanol, 1,2-propanediol, 1,3-propanediol, 1,3-butanediol, 1,4-butanediol, 1,2-hexanediol, 1,6-hexanediol, glycerol, benzyl alcohol, phenoxyethanol and phenylethyl alcohol.

3. The agent according to claim 2, wherein, based on the total weight of the agent, the agent comprises the one or more alcohols (a) in a total amount of at least about 25.0% by weight.

4. The agent according to claim 2, wherein, based on the total weight of the agent, the agent comprises (a) at least about 30.0% by weight.

5. The agent according to claim 1, wherein, based on the total weight of the agent, the agent has a water content between from 0 and about 8% by weight.

6. The agent according to claim 1, wherein the agent:
(b) the at least one clay mineral modified with quaternary ammonium compounds is chosen from one or more of montmorillonite, sepiolite, hectorite, palygorskite, vermiculite, glauconite, saponite, beidellite, nontronite, sauconite, bentonite and stevensite, most preferably selected from montmorillonite and sepiolite.

7. The agent according to claim 1, wherein the agent comprises as component (b):
(bi) at least one first clay mineral modified with quaternary ammonium compounds, wherein the clay mineral is selected from sepiolite and/or palygorskite, and
(bii) at least one second clay mineral modified with quaternary ammonium compounds, wherein the clay mineral is a clay mineral of the smectite type, more preferably montmorillonite.

8. The agent according to claim 1, wherein the agent comprises as component (b):
(bi) a first clay mineral modified with quaternary ammonium compound, wherein the clay mineral is sepiolite, and
(bii) a second clay mineral modified with quaternary ammonium compound, wherein the clay mineral is montmorillonite, and
(biii) a third clay mineral modified with a quaternary ammonium compound, wherein the clay mineral is hectorite.

9. The agent according to claim 1, wherein, based on the total weight of the agent, the agent:
(b) the one or more clay minerals modified with quaternary ammonium compounds in a total amount of from about 0.1 to about 10.0% by weight.

10. The agent according to claim 1, wherein the agent comprises as the at least one color pigment (c) at least one inorganic color pigment that is chosen from one or more of colored metal oxides, metal hydroxides, metal oxide hydrates, silicates, metal sulfides, complex metal cyanides, metal sulfates, bronze color pigments and/or from colored pigments based on mica which are coated with at least one metal oxide and/or one metal oxychloride.

11. The agent according to claim 1, wherein the at least one color pigment (c) is a colored pigment based on mica, which is coated with one or more metal oxides from the group of titanium dioxide (CI 77891), black iron oxide (CI 77499), yellow iron oxide (CI 77492), red and/or brown iron oxide (CI 77491, CI 77499), manganese violet (CI 77742), ultramarines (sodium aluminum sulfosilicates, CI 77007, color pigment Blue 29), chromium oxide hydrate (CI 77289), chromium oxide (CI 77288) and/or iron blue (Ferric Ferrocyanide, CI 77510).

12. The agent according to claim 1, wherein, based on the total weight of the agent,
component (c) comprises the at least one color pigments in a total amount of from about 1.0 to about 25.0% by weight.

13. The agent according to claim 1, wherein the agent further comprises one or more oils chosen from: isopropyl myristate, isopropyl laurate, isopropyl palmitate, isopropyl stearate, 2-ethylhexyl laurate, 2-ethylhexyl myristate, 2-ethylhexyl palmitate, 2-ethylhexyl cocoate, 2-ethylhexyl stearate, 2-ethylhexyl isostearate, hexyldecyl laurate, hexyldecyl stearate, isooctyl stearate, isononyl isononanoate, isononyl stearate, isotridecylnonanoate, 2-octyldodecyl palmitate and isocetyl stearate.

14. The agent according to claim 1, wherein the total amount of the anionic polymers included in the agent, based on the total weight of the agent, is below about 2.5% by weight.

15. (canceled)

16. The agent according to claim 2, wherein, based on the total weight of the agent, the agent comprises the one or more alcohols (a) in a total amount of at least about 3 5.0% by weight.

17. The agent according to claim 2, wherein, based on the total weight of the agent, the agent comprises the one or more alcohols (a) in a total amount of at least about 40.0% by weight.

18. The agent according to claim 2, wherein, based on the total weight of the agent, the agent comprises the one or more alcohols (a) in a total amount of at least about 50.0% by weight.

19. The agent according to claim 1, wherein, based on the total weight of the agent, the agent has a water content between from 0 and about 3% by weight.

20. A method for the temporary dyeing of keratinic materials, wherein the method comprises the following method steps:
a) providing an agent in a pump-spray container or an aerosol-spray container, wherein the agent comprises:
(1) at least one aliphatic and/or aromatic alcohol having from 2 to about 8 carbon atoms,
(2) at least one clay mineral modified with quaternary ammonium compounds, and
(3) at least one color pigment, and
b) applying the agent to the keratinic materials.

21. The method of claim 20, wherein the step b) applying comprises spraying.
